(19) United States
(12) Patent Application Publication
(10) Pub. No.: US 2024/0307691 A1
(43) Pub. Date: Sep. 19, 2024

(54) MEDICAL SYSTEMS FOR MANAGING CRITICAL EVENTS

(71) Applicant: Boston Scientific Neuromodulation Corporation, Valencia, CA (US)

(72) Inventors: Lisa Denise Moore, Glendale, CA (US); Chirag Shah, Valencia, CA (US); Sarah Lysogorski, Santa Monica, CA (US)

(21) Appl. No.: 18/597,477

(22) Filed: Mar. 6, 2024

Related U.S. Application Data
(60) Provisional application No. 63/451,712, filed on Mar. 13, 2023.

(51) Int. Cl. A61N 1/36; A61N 1/05; G16H 20/40; G16H 40/63; G16H 50/20; G16H 50/30; G16H 80/00

(52) U.S. Cl. CPC A61N 1/36139; A61N 1/0534; A61N 1/36071; G16H 20/40; G16H 40/63; G16H 50/20; G16H 50/30; G16H 80/00

(57) ABSTRACT

A system may include medical device(s) and a processing system used to determine that at least one event experienced by the patient is at least one critical event, determine a severity of each critical event and weight each critical event based on the determined severity to provide a corresponding weighted critical event, sum the weighted critical event, determine an overall clinical priority for the patient based at least in part on the sum and weight the patient based on the determined overall clinical priority, select a communication technique for use to communicate the at least one critical event based on the weighted patient priority, and communicate the critical event using the selected communication technique.

### CLAIM OF PRIORITY

This application claims the benefit of U.S. Provisional Application No. 63/451,712 filed on Mar. 13, 2023, which is hereby incorporated by reference in its entirety.

### TECHNICAL FIELD

This document relates generally to medical systems, and more particularly, but not by way of limitation, to systems, devices, and methods for identifying critical therapy events (e.g., red flag events), providing patient management to appropriately address the critical therapy events, and providing appropriate notices of the critical therapy events.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS). Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

Many factors may be considered to configure the therapy device to treat the condition. For example, the condition being treated by a DBS therapy may be associated with many direct and indirect symptoms, and it is desirable to configure the DBS therapy to positively affect the symptoms associated with the condition while avoiding or reducing any possible side effects. Further, patients may be prescribed medicine to treat the condition being treated with the DBS therapy, or to treat another disease or condition of the patient. These prescriptions may change which may change the direct and/or indirect symptoms associated with the condition being treated with the DBS therapy. The direct and indirect symptoms of the condition may also change because of disease progression and other changes to the patient's overall health. Therefore, given all of the potential reasons why the symptoms change, it can be difficult to interpret the changes. For example, it can be difficult to determine whether the DBS therapy may be modified to improve the patient's condition. Improvements are desired to identify and appropriately manage critical events (e.g., red flag events) and provide appropriate notices of the critical events.

### SUMMARY

An example (e.g., “Example 1”) of a system may include at least one medical device and a processing system. The medical device(s) may be configured to treat a condition by delivering a therapy to a patient. The therapy may be at least partially defined using a parameter set. The processing system may be used to determine that at least one event experienced by the patient is at least one critical event where each of the at least one critical event is one of a plurality of predefined critical events, determine a severity of each of the at least one critical event, and weight each of the at least one critical event based on the determined severity to provide a corresponding at least one weighted critical event, sum the at least one weighted critical event to provide a sum of at least one weighted critical event, determine an overall clinical priority for the patient based at least in part on the sum, and weight the patient based on the determined overall clinical priority to provide a weighted patient priority, select a communication technique from a plurality of available communication techniques based on the weighted patient priority, and communicate the critical event using the selected communication technique.

In Example 2, the subject matter of Example 1 may optionally be configured such that the processing system is further used to adjust the weighted patient priority based on whether corrective action is successfully performed by the medical device to address the at least one critical event, and the communication technique is selected based on the adjusted weighted patient priority.

In Example 3, the subject matter of Example 2 may optionally be configured such that the corrective action is performed by revising a current program for the medical device, reverting to a prior program for the medical device, replacing the current program with another program for the medical device, or toggling programs for the medical device.

In Example 4, the subject matter of any one or more of Examples 1-3 may optionally be configured such that the medical device includes a deep brain stimulator (DBS) and the therapy includes a DBS therapy.

In Example 5, the subject matter of Example 4 may optionally be configured such that the at least one critical event is specific to the condition of the patient, specific to the DBS therapy, or specific to the medical device used to deliver the therapy.

In Example 6, the subject matter of any one or more of Examples 1-5 may optionally be configured such that the severity of each of the at least one critical event is determined using a plurality of weight factors that include at least two of the following: whether the corresponding critical event is due to a newly recommended stimulation setting that the patient applied remotely, whether the corresponding critical event is potentially life threatening, whether the corresponding critical event is anticipated, the regularity of an occurrence for the corresponding critical event based on a pre-assessed normal occurrence of the critical event for the patient, or a comparison of a severity score for the corresponding critical event to a pre-assessed normal for the patient.

In Example 7, the subject matter of any one or more of Examples 1-6 may optionally be configured such that the overall clinical priority for the patient is determined using a combination of the sum, as well as a determination that one or more of the at least one critical event is likely related to a change in medication or stimulation.

In Example 8, the subject matter of any one or more of Examples 1-7 may optionally be configured such that the overall clinical priority for the patient is determined using a combination of the sum, as well as when the patient is scheduled for a clinical visit.

In Example 9, the subject matter of any one or more of Examples 1-8 may optionally be configured such that the weighted patient priority is determined to be high based on a single weighted critical event or based on a combination of two or more weighted critical events.

In Example 10, the subject matter of any one or more of Examples 1-9 may optionally be configured such that the processing system is configured to monitor for the at least one event within an observation window to set for a time after a treatment change.

In Example 11, the subject matter of any one or more of Examples 1-10 may optionally be configured such that the overall clinical priority is attributable to a treatment change and a determination whether the at least one critical event is life threatening or non-life-threatening.

In Example 12, the subject matter of any one or more of Examples 1-10 may optionally be configured such that the determined overall clinical priority is attributable to patient deterioration rather than the treatment change or an acute event.

In Example 13, the subject matter of any one or more of Examples 1-10 may optionally be configured such that the determined overall clinical priority is attributable to a need for replacing the medical device.

In Example 14, the subject matter of any one or more of Examples 1-13 may optionally be configured to further include a device with a downloadable app, wherein the plurality of available communication techniques for use to communicate the at least one critical event includes at least two techniques selected from a phone call, a text message, an email message, a patient-connected app configured to provide alerts on the device, where the alerts include one or more of a badge, a banner or a sound alert. By way of example and not limitation, the device may be a personal device such a phone or tablet, or may be a device provided by a medical device company.

In Example 15, the subject matter of any one or more of Examples 1-14 may optionally be configured such that the plurality of available communication techniques for use to communicate the at least one critical event includes at least one of a device rep portal or a clinician/physician portal.

Example 16 includes subject matter (such as a method, means for performing acts, machine readable medium including instructions that when performed by a machine cause the machine to perform acts, or an apparatus to perform). The subject matter may include using a medical device configured to treat a condition by delivering a therapy to a patient. The therapy may be at least partially defined using a parameter set. The subject matter may further include using a processing system to determine that at least one event experienced by the patient is at least one critical event where each of the at least one critical event being one of a plurality of predefined critical events, determine a severity of each of the at least one critical event, and weight each of the at least one critical event based on the determined severity to provide a corresponding at least one weighted critical event, sum the at least one weighted critical event to provide a sum of at least one weighted critical event, determine an overall clinical priority for the patient based at least in part on the sum, and weight the patient based on the determined overall clinical priority to provide a weighted patient priority, select a communication technique from a plurality of available communication techniques based on the weighted patient priority, and communicate the critical event using the selected communication technique.

In Example 17, the subject matter of Example 16 may optionally be configured such that the processing system is further used to adjust the weighted patient priority based on whether corrective action is successfully performed by the medical device to address the at least one critical event, and the communication technique is selected based on the adjusted weighted patient priority.

In Example 18, the subject matter of Example 17 may optionally be configured to further include performing the corrective action to address the at least one critical event, where the corrective action includes revising a current program, reverting to a prior program, replacing the current program with another program, or toggling programs.

In Example 19, the subject matter of any one or more of Examples 16-17 may optionally be configured such that the medical device includes a deep brain stimulator (DBS) and the therapy includes a DBS therapy.

In Example 20, the subject matter of Example 19 may optionally be configured such that the at least one critical event is specific to the condition of the patient, specific to the DBS therapy, or specific to the medical device used to deliver the therapy.

In Example 21, the subject matter of any one or more of Examples 16-20 may optionally be configured such that the severity of each of the at least one critical event is determined using a plurality of weight factors that include at least two of the following: whether the corresponding critical event is due to a newly recommended stimulation setting that the patient applied remotely; whether the corresponding critical event is potentially life threatening; whether the corresponding critical event is anticipated; the regularity of an occurrence for the corresponding critical event based on a pre-assessed normal occurrence of the critical event for the patient; or a comparison of a severity score for the corresponding critical event to a pre-assessed normal for the patient.

In Example 22, the subject matter of any one or more of Examples 16-21 may optionally be configured such that the overall clinical priority for the patient is determined using a combination of the sum, as well as at least one of: a determination that one or more of the at last one critical event is likely related to a change in medication or stimulation; and when the patient is scheduled for a clinical visit.

In Example 23, the subject matter of any one or more of Examples 16-22 may optionally be configured such that the weighted patient priority is determined to be high based on a single weighted critical event or based on a combination of two or more weighted critical events.

In Example 24, the subject matter of any one or more of Examples 16-23 may optionally be configured to further include monitoring for the at least one event within an observation window to set for a time after a treatment change.

In Example 25, the subject matter of any one or more of Examples 16-24 may optionally be configured such that the determined overall clinical priority is attributable to: a treatment change and a determination whether the at least one critical event is life threatening or non-life-threatening, a patient deterioration rather than the treatment change or an acute event, or a need for replacing the medical device.

In Example 26, the subject matter of any one or more of Examples 16-25 may optionally be configured such that the plurality of available communication techniques for use to communicate the at least one critical event includes at least two techniques selected from a phone call, a text message, an email message, phone alerts including at least one of badges, banners or sound, a patient-connected app, a device rep portal and a clinician/physician portal.

Example 27 includes subject matter (such as a method, means for performing acts, machine readable medium including instructions that when performed by a machine cause the machine to perform acts, or an apparatus to perform). The subject matter may include determining that at least one event experienced by a patient is at least one critical event, each of the at least one critical event being one of a plurality of predefined critical events where the patient is receiving a therapy delivered using a medical device to treat a condition, determining a severity of each of the at least one critical event, and weight each of the at least one critical event based on the determined severity to provide a corresponding at least one weighted critical event, summing the at least one weighted critical event to provide a sum of at least one weighted critical event, determining an overall clinical priority for the patient based at least in part on the sum, and weighting the patient based on the determined overall clinical priority to provide a weighted patient priority, selecting a communication technique from a plurality of available communication techniques for use to communicate the at least one critical event, the communication technique being selected based on the weighted patient priority, and communicating the critical event using the selected communication technique.

In Example 28, the subject matter of Example 27 may optionally be configured to further include adjusting the weighted patient priority based on whether corrective action is successfully performed by the medical device to address the at least one critical event, wherein the communication technique is selected based on the adjusted weighted patient priority.

In Example 29, the subject matter of any one or more of Examples 27-28 may optionally be configured such that the medical device includes a deep brain stimulator (DBS) and the therapy includes a DBS therapy.

In Example 30, the subject matter of Example 29 may optionally be configured such that the at least one critical event is specific to the condition of the patient, specific to the DBS therapy, or specific to the medical device used to deliver the therapy.

In Example 31, the subject matter of any one or more of Examples 27-30 may optionally be configured such that the severity of each of the at least one critical event is determined using a plurality of weight factors that include at least two of the following: whether the corresponding critical event is due to a newly recommended stimulation setting that the patient applied remotely; whether the corresponding critical event is potentially life threatening; whether the corresponding critical event is anticipated; the regularity of an occurrence for the corresponding critical event based on a pre-assessed normal occurrence of the critical event for the patient; or a comparison of a severity score for the corresponding critical event to a pre-assessed normal for the patient.

In Example 32, the subject matter of any one or more of Examples 27-31 may optionally be configured such that the overall clinical priority for the patient is determined using a combination of the sum, as well as at least one of: a determination that one or more of the at last one critical event is likely related to a change in medication or stimulation; and when the patient is scheduled for a clinical visit.

In Example 33, the subject matter of any one or more of Examples 27-32 may optionally be configured such that the weighted patient priority is determined to be high based on a single weighted critical event or based on a combination of two or more weighted critical events.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIG. 8 illustrates, by way of example and not limitation, a method for identifying, rating, and reacting intelligently to critical events that occur in DBS patients.

FIG. 9 provides another illustration, by way of example and not limitation, a method for identifying, rating, and reacting intelligently to critical events that occur in DBS patients.

FIG. 10 illustrates, by way of example and not limitation, a method for revising a current program to treat motor symptoms, by adjusting amplitude.

FIG. 11 illustrates, by way of example and not limitation, a method for identifying and addressing worsening of the patient's condition related to a treatment change.

FIG. 12 illustrates, by way of example and not limitation, a method for identifying and addressing patient deterioration.

FIG. 13 illustrates, by way of example and not limitation, a method for identifying and addressing a need for a stimulator replacement.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

In medicine, red flag or critical events indicate the potential presence of significant health complications that the clinician should address. Embodiments of the present subject matter include medical systems used to deliver a therapy to treat a condition of the patient. The medical systems may use a red flag concept for events specific to the therapy, specific to the patient's condition, and/or specific to a device or devices used to deliver the therapy. For example, red flag alerts may be prominently displayed to patient and caregiver applications and physician portals. A portal may be a web-based platform that provides an audience with access to information. Different audiences may use different portals. Embodiments of the present subject matter provide a system capable of identifying, rating, and reacting intelligently to critical events that occur in DBS patients. Reactions may include suggesting a patient return to a previous setting or informing the patient and those with permissions that a clinical follow up may be needed.

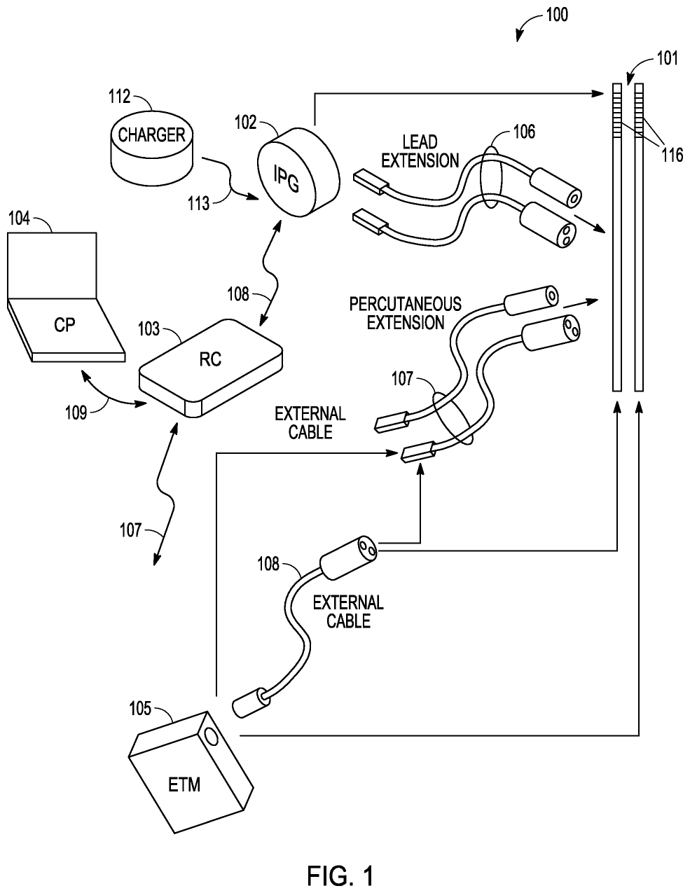

The present subject matter relates to FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

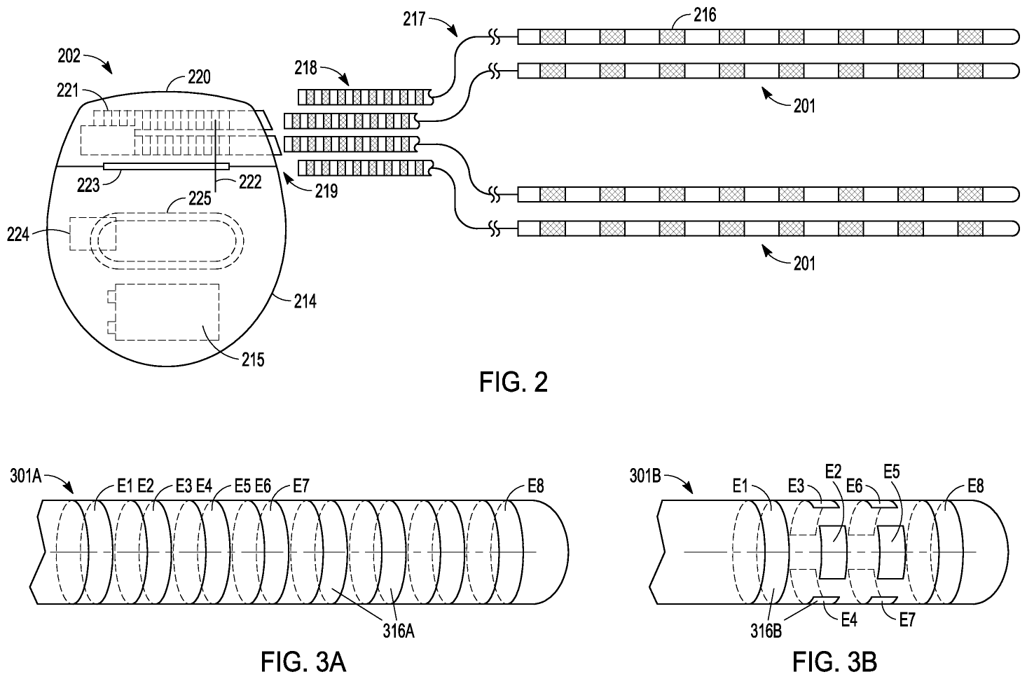

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 225 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead 219.

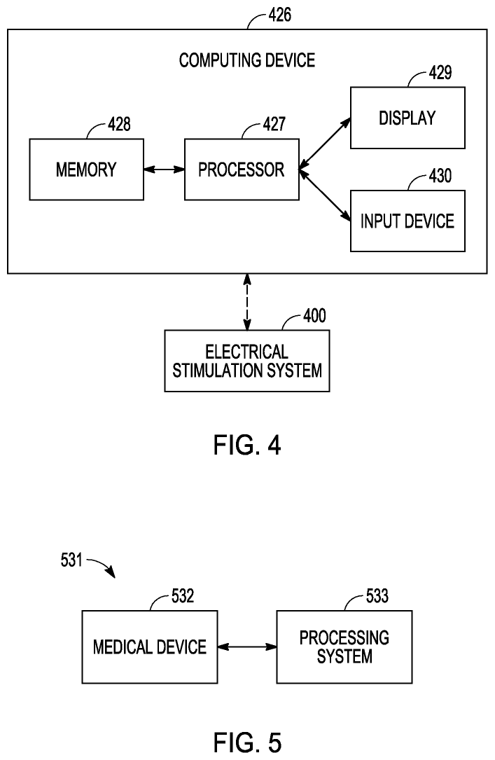

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, that is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to provide sensing functions and/or therapy functions. For example, the medical device may include a device configured to use a parameter set to deliver an electrical stimulation therapy. The medical device may be an implantable medical device such as an implantable neurostimulator. The implantable medical device may be configured to deliver SCS or DBS therapy. The medical device may include more than one medical device. The processing system may be within a single device, or may be a distributed system across two or more devices including local and/or remote systems. According to various embodiments, the medical system may include at least one medical device configured to treat a condition by delivering a therapy to a patient. The medical system may include a processing system configured for use to determine that at least one event experienced by the patient is at least one critical event. The processing system may determine a severity of each of the at least one critical event, and weight each of the at least one critical event based on the determined severity to provide a corresponding at least one weighted critical event. The processing system may sum the at least one weighted critical event to provide a sum of at least one weighted critical event, determine an overall clinical priority for the patient based at least in part on the sum, and weight the patient based on the determined overall clinical priority to provide a weighted patient priority. The processing system may be configured to adjust the weighted patient priority when corrective action is successfully performed by the medical device to address the at least one critical event. The processing system may select a communication technique from a plurality of available communication techniques for use to communicate the at least one critical event where the communication technique being selected based on the weighted patient priority. The critical event may be communicated using the selected communication technique.

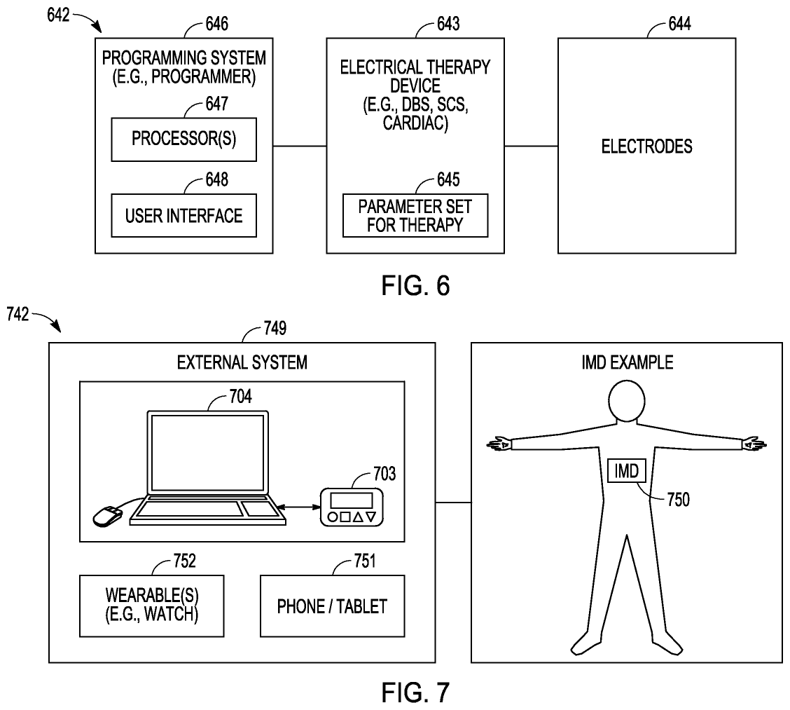

FIG. 6 illustrates, by way of example, an example of an electrical therapy-delivery system. The illustrated system 642 may be a more specific example of the system illustrated in FIG. 5, or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, that may include one or more processors 647 and a user interface 648. The programming system 646 may be used to program and/or evaluate the parameter set(s) used to deliver the therapy. The illustrated system 642 may be a DBS system.

In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 7 illustrates, by way of example and not limitation, the electrical therapy-delivery system of FIG. 6 implemented using an IMD. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote control device 703, similar to RC 103 in FIG. 1, configured for use by the patient to communicate with and program the neuromodulator. For example, the remote control device 703 may allow the patient to turn a therapy on and off, change or select programs, and/or may allow the patient to adjust patient-programmable parameter(s) of the plurality of modulation parameters. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. Notifications may be sent to the patient, physician, device rep or other users via the external system and through remote portals (e.g., web-based portals) provided by remote systems.

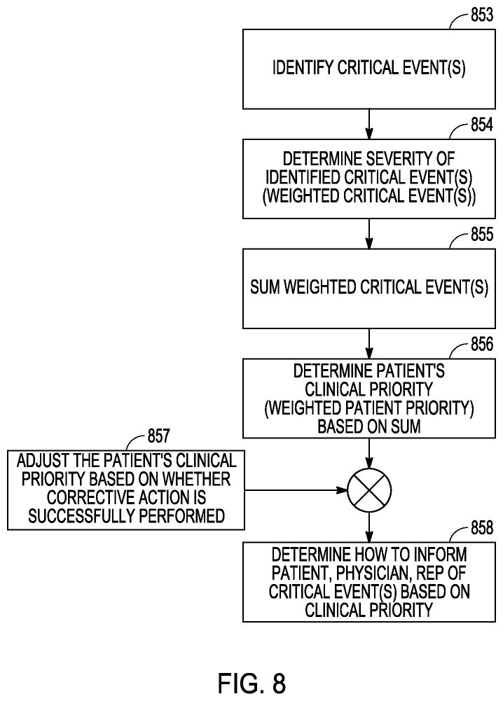

FIG. 8 illustrates, by way of example and not limitation, a method for identifying, rating, and reacting intelligently to critical events that occur in DBS patients. A medical device may be used to deliver a DBS therapy to a patient. Similar methods may be performed for therapies other than DBS. A processing system may be configured identify critical event(s) 853. For example, at least one event experienced by the patient may be determined to be a critical event. The critical event(s) may be selected from a predefined list of prioritized events. At 854 the severity of the identified critical event(s) may be determined and the critical event(s) may be weighted based on the determined severity to provide a corresponding at least one weighted critical event. Severity may be used to weight the event and may be calculated based on an assessed threat to patient well-being, degree of worsening, and degree of positive improvement. At 855, one or more weighted events are summed together. The weighted critical event(s) may correspond to an observation window of time. The observation window may be a moving window, such that all weighted events that fall within the window are summed together. By way of example and not limitation, the observation window may be a two-week (e.g., 14 day) period of time that refreshes every day such that the period of time is over days 1-14 on the 14th day, and is over days 2-15 on the 15th day, and the like. Other periods of time (e.g., number of days, hours, minutes, etc.) may be used to provide an observation window in which the weighted critical events are summed. At 856, an overall clinical priority for the patient may be determined based at least in part on the sum. The patient may be weighted (e.g., prioritized) based on the determined overall clinical priority to provide a weighted patient priority. Thus, the patient is triaged based on an assessment of one or more events over the observation window. One patient's need for action may be weighted against another via communication through a physician portal. It may be determined if immediate corrective action can be taken via an automated review of different setting combinations available on device and an estimated outcome of a change. As illustrated at 857 the patient's clinical priority may be adjusted based on whether corrective action can be successfully performed to address the critical event(s). For example, a clinical priority may be lowered if corrective action can be successfully taken. At 858, it may be determined, based on the clinical priority or adjusted clinical priority, how to inform the patient physician, or device rep of the critical event(s) (e.g., via different portals, apps, phone alerts, etc.). For example, a communication technique may be selected from a plurality of available communication techniques for use to communicate the critical event(s). Patient and connected apps may be informed based on the weighted need and the ability/inability to resolve with corrective action. The communication technique may be selected based on the weighted patient priority. The critical event may be communicated using the selected communication technique.

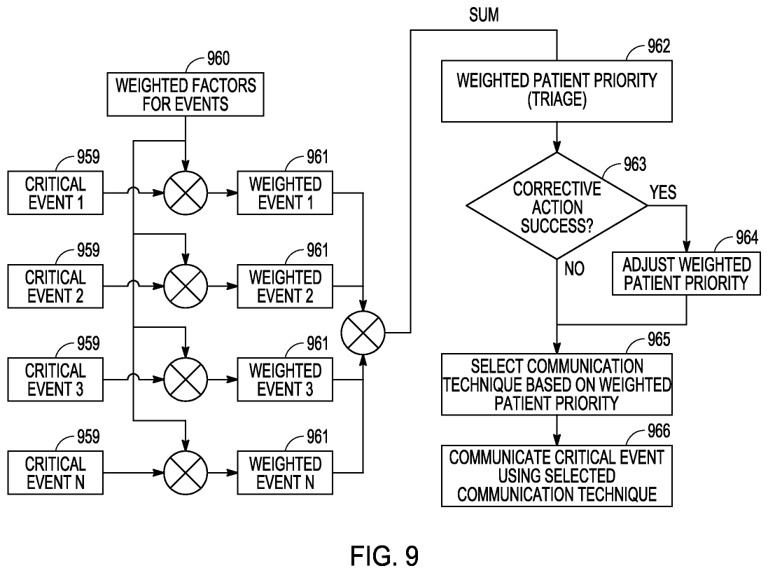

FIG. 9 provides another illustration, by way of example and not limitation, a method for identifying, rating, and reacting intelligently to critical events that occur in DBS patients. One or more critical events 959 (e.g., Critical Events 1-N) may be identified within an observation window. The observation window may be a moving window. Weight factors 960 may be applied to each of the identified critical events to provide one or more weighted events 961 (e.g., Weighted Events 1-N). The weighted events, within the observation window, may be summed together to provide a weighted patient priority 962 used to triage one patient with respect to other patients. A determination may be automatically performed to determine whether a corrective action is capable of being automatically performed. At 963, the method may determine whether the corrective action is a success. If the corrective action is successful, the weighted patient priority may be adjusted 964. As illustrated at 965 a communication technique is selected based on the weighted patient priority. Different communication techniques may include different audiences for the communication (e.g., patient, doctor or clinician, caregiver, and/or device), may include different channels of communication to a person (e.g., phone call, text, email, portal messages or reports, app messages, phone alerts such as badges, banners, sounds), and different timing for the communications (e.g., immediate communication or alert, or a report or group of alerts that may periodically be provided such as, but not limited to daily, weekly or monthly reports. The different communication techniques may also involve push techniques where the system pushes communication to the desired the desired audience, or pull techniques where the audience requests the information from the system. At 966 a critical event is communicated using the selected communication technique.

Critical events 959 for DBS patients may be events that can be disease or treatment specific. By way of example and not limitation, critical events 959 may include events selected from predefined events. Mood changes may be detected using questionnaires. Vision and/or speech difficulties may be detected using questionnaires or the patient performing specific tasks. The worsening of dyskinesia or treatment induced side effects may be determined using questionnaires, specific tasks, sensed signals internal or external signals. The worsening of disease specific symptoms (e.g., tremor, bradykinesia, etc.) may be determined using questionnaires, specific tasks, internal sensed signals or external sensed signals. The worsening of disease associated symptoms (e.g., blood pressure, constipation, etc.) may be determined using questionnaires, specific tasks, internal sensed signals or external sensed signals. A significant decrease in activity may be determined using questionnaires, specific tasks, internal sensed signals or external sensed signals. Increased falls may be determined using questionnaires, specific tasks, internal sensed signals or external sensed signals. A stimulator being OFF for an extended period may be determined using questionnaires or IPG downloaded data. Increased medication intake that is not prescribed may be determined using questionnaires, internal or external sensed signals with a recognition algorithm. A high degree of programming changes made by the patient outside of clinical visit or recommended stimulation changes may be determined using questionnaires or IPG downloaded data. Patient disengagement or significant deviation from patient's standard behavior may be determined by a change in an overall utilization of provided tools (e.g., interaction with an app or a patient remote control). A patient's dislike of a newly applied recommended stimulation setting (done remotely) may be determined by questionnaires or by downloaded data indicative of monitored usage of the recommended setting. System issues such as End Of Life of the primary cell or patterns in charging may be detected using questionnaires or IPG downloaded data. Some or all of the above-mentioned information may be provided by a secondary individual (such as a caregiver) to whom the patient has consented to provide such information.

The weight factors 960 may be based on a determined severity of the individual event. The severity of a single event may be determined using several factors. The event may be weighted based on whether it is due to a newly recommended setting that the patient applied remotely. The event may be weighted based on whether it is potentially life threatening. The event may be weighted based on how severe the event is based on the patient's normal (e.g., comparing a scored event to a normal score). The event may be weighted based on how regular the event is or has recently been occurring based on the normal occurrence for the event for that patient. The event may be weighted based on whether it is an anticipated event (e.g., an expected side effect to a treatment change). Sometimes when treatment changes are made the physicians must balance improving symptoms with some minor treatment induced side effects. Any anticipated event will be lowered in severity rating. By way of a fall event example, a single fall event may start with a higher severity because it is considered a life-threatening event. However, if a fall event occurs once over a several week or month span, then the occurrence may be considered low and therefore the severity is lowered. In another example related to mood, a patient's answers to mood related questionnaires may indicate a mood change. The event may be weighted high if the mood change indicates an increase in depression, or may be weighted low if it shows a repetitive behavior. The more times this occurs the higher the severity.

The patient may be triaged by calculating or otherwise determining the patient's overall clinical priority. This may be used within a physician portal to weight one patient's need for action against another (physician portal). As reps may assist with treatment management for some conditions, the same or similar triage information may be presented via a rep portal. Determining the patient's clinical priority may be based on several factors. One factor is whether the event is likely related to a recent treatment change (medication or stimulation). An observation window after a treatment change may be used to monitor for changes, and all negative changes within the window may be weighted higher for a set time and interaction expectation. One factor is when the patient is next scheduled for a clinical visit. One factor is the total events experienced and the severity of the events. The severity may be identified using a point rating scale. All events are considered together. A single high-level event may create a significant clinical priority, or multiple mid-level events, in the absence of an improvement in positive outcomes, may add up to a significant clinical priority

The post-treatment change observation window may be similar to what may be seen in a clinical setting where the patient is observed for a set period of time after a treatment change is made to ensure that the treatment change is improving symptom outcome without serious side effects. Some symptoms and side effects take longer to wash in than the time available in clinic. By way of example and not limitation, the observation window may be set for a specified period of time (e.g., two weeks) during which symptoms that take time to wash in would be observed. The observation window may be specific to the anticipated wash in time or occurrence probability of specific events. For example, falls may be critically observed for a month while mood may be for two weeks and speech may be one week. The observation window may be extended if patient interaction with required reporting metrics (app, sensors, etc.) is low.

Various embodiments may determine if immediate corrective action can be taken to address the events. For example, different setting combinations that are available on the medical device may be automatically reviewed to determine if an estimated outcome for changing will appropriately address the event. If the event can be appropriately addressed, corrective action can be taken immediately rather than waiting for a clinical visit. The corrective action may include adjusting a current program, reverting to the settings originally (or more generally previously) programmed on a given program, replacing a program, or switching to another program. Corrective action may be performed by revising a current program for the medical device, reverting to a prior program for the medical device, replacing the current program with another program for the medical device, or toggling programs for the medical device.

The physician may be informed about a corrective action that was performed, provided the patient has consented to share their information. If a corrective action is successful, the clinical priority may be recalculated (reduced), and the physician may see a summary of patients who have received corrective actions successfully. If a corrective action is unsuccessful, the clinical priority may be recalculated (increased).

A corrective action may involve revising a current program. Often clinicians will recommend modest changes to improve patient outcomes. Generally, the program may be revised to avoid side effects while also improving the efficacy of the therapy. The program may be revised by revising various stimulation parameters, such as amplitude, pulse width, pulse frequency, duty cycle, electrode fractionalization, and the like.

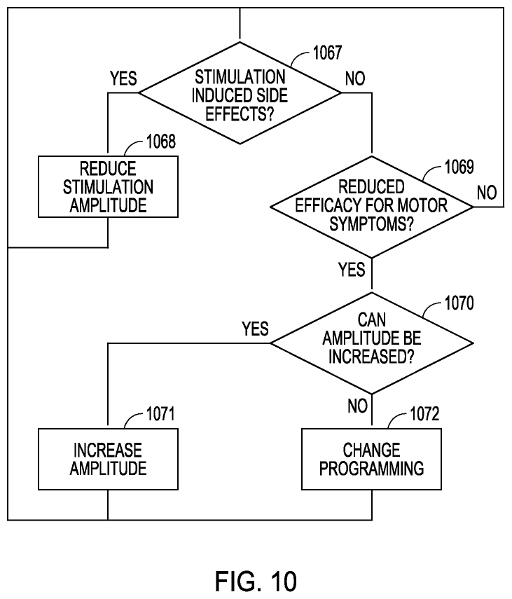

FIG. 10 illustrates, by way of example and not limitation, a method for revising a current program to treat motor symptoms, by adjusting amplitude. At 1067, it is determined whether the stimulation provided by the current program is inducing side effects. The stimulation amplitude may be reduced at 1068 to avoid the side effect, and the process may return to 1067 to determine if side effects are still being induced. If side effects are not being induced and if the stimulation of the current program has reduced efficacy for motor systems 1069, the process may determine whether amplitude can be increased 1070. If available, amplitude may be increased at 1071 in an attempt to improve efficacy. If amplitude cannot be increased, then the program may be changed at 1072. Based on the critical events identified the flow on the right (1069, 1070, and 1071 or 1072) will be followed except for when it is determined to revert the program. If the changes are efficacious this flow may be automatically generated into a schedule. If the changes are not efficacious or short lived, then the next corrective action may be attempted.

A corrective action may involve reverting a program. Some conditions may trigger a revert process to restore a previous program stored in memory. When a programming setting has been changed, the previous settings may be stored. For example, settings may only be stored for a potential reversion process if they have been applied for a certain minimum amount of time. By way of example and not limitation, the program may need to be implemented for one week before it can be stored for a potential reversion. The revert option may be used automatically in the instance that a patient has accepted a stimulation recommendation, which was applied remotely, and does not like the new setting, or in the instance that the stimulation-induced side effect is a mood change that occurred after the stimulation fractionalization was changed. The revert option may be used during the post-treatment change observation window if no, or minimal, disease specific medication change was made. The patient may need to replace their program if the revert option is not available.

A corrective action may involve creating a replacement program when the program cannot be revised or reverted. For example, previously-trialed stimulation settings that have shown good efficacy (clinical benefits) without recording of side effects may be applied to the patient. Alternatively, settings can be trialed that were never applied but are determined to be in a safe search space for the different stimulation parameters. As is done in clinic, the amplitude of the stimulation setting may be slowly increased (ramped) allowing the patient to pause and provide feedback manually or to be collected automatically (internal/external sensing) to verify that stimulation induced side effects are not occurring. The ramping up of the stimulation may stop when the target is achieved or the side effect is discovered. Multiple settings can be tested in this way with the best setting (as determined by the feedback) being applied. The creation of the replacement program may be conducted similar to the way an in-person programming session is done except no physician needs to be involved.

A corrective action may involve toggling a program schedule. For example, if revising, reverting or replacing the program does not work, then it may be assumed the patient may have habituated to the current settings if the patient is not under an observation window and the patient is a set time out from their last treatment change. A few programs (e.g., 2 or 3 programs) from the replacement program process may be applied in a schedule such that each program runs for a moderate length (a few minutes, a few hours, a few days, a week) before switching to another program. For example, the programs may initially be switched, as part of a trial, every day with the patient's consent. If unresolved and no/minimal additional stimulation induced side effects are observed, this time may be reduced up to the set limit.

Various embodiments determine how to inform the patient, physician, caregiver and/or device reps, which may be based on the weighted need and/or based on whether correction action was successful. Examples of available communication techniques that may be selected for use to communicate the event(s) may include various combinations of a phone call, a text message, an email message, phone alerts including at least one of badges, banners or sound, a patient-connected app, a device rep portal and a clinician/physician portal. The various techniques may include push communication technique and pull communication techniques.

A device rep may always be informed of patient's clinical priority rating. The clinician will always see the patient's clinical priority rating if the patient has chosen to share the data. This rating will be visually distinct to encourage the physician to quickly triage if it falls within predefined thresholds. The patient may be informed when their clinical priority exceeds a predefined threshold and recommended to speak to their doctor. If the patient shared data with a caregiver and given appropriate permissions to the caregiver, the caregiver may see the patient's clinical priority at this time and be recommended to talk to the patient's doctor. If the patient has not shared data with the doctor, the patient may be prompted to share all or part of their data again along with a justification (e.g., you may need a treatment change).

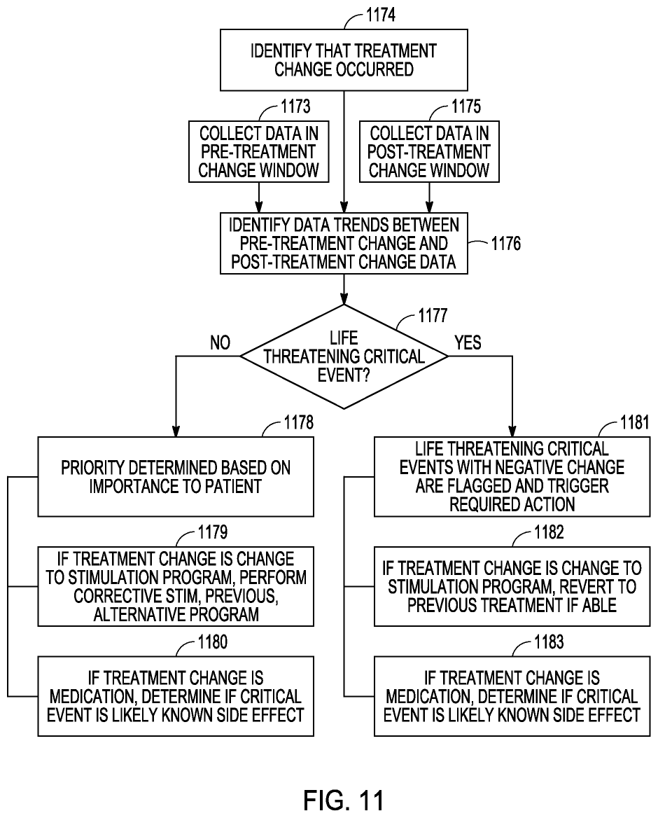

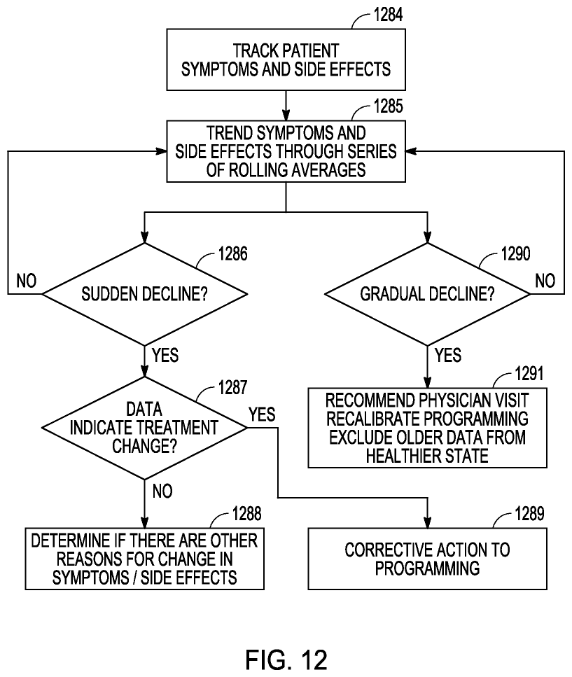

FIG. 11 illustrates, by way of example and not limitation, a method for identifying and addressing worsening of the patient's condition related to a treatment change. Data may be collected, and stored for a period of time corresponding to a rolling time window (e.g., a pre-treatment window of a plurality of epochs) 1173. The plurality of epochs may correspond to a predefined number of weeks, days, hours or minutes. For example, the pre-treatment window of data collection may collect the last seven days of data. A treatment change may be identified 1174, and data may be collected in a post-treatment change window of another number of epochs 1175. It may be determined whether the change is a stimulation treatment change, a medication treatment change or both. This determination may be made from an automatic download of a stimulation log, a patient questionnaire, or a physician portal notification. Determine whether treatment change is to stimulation, medication, or both. A timepoint may be flagged as a Treatment Change timepoint.

Pre-treatment change data may be collected for each critical event feature, and may be analyzed for features including average and standard deviation. Data may only be included within the window if the treatment conditions are consistent. Data may be collected from combination of internal sensors, external sensors, questionnaires, etc.

A treatment window may be set from the time of treatment change to the anticipated time to observe change. The post-treatment change observation window for motor events may be limited to two weeks. Non-motor events are up to 1 month. Critical events that may be life threatening (blood pressure, cardiac, falls, depression, etc.) may be considered independently.

At 1176 the pre-treatment change data 1173 and the post-treatment change data 1175 may be compared to identify data trends 1176. The identified data trends may include changes in mean, median, or significant outliers. The degree of negative change may be multiplied by the weight of the critical event.

Life threatening versus non-life-threatening events are flagged in the system 1177. Non-life-threatening events may be weighted such that symptoms that are more important to the patient receive a higher priority and are multiplied against the change 1178. The result may be summed and if a threshold of negative change is met then a treatment change is recommended. If the treatment change is only a stimulation change, a corrective stimulation action may be performed on the current program and the patient may be observed for set time; and if no improvement observed, a previous or alternative program may be tried 1779. If a medication change is included, it may be determined whether the critical event is more likely the result of a known medication side effect 1180. If the critical event is likely a medication side effect, the patient may be observed for another month, and a physician/rep may be notified to review if there is not improvement. Given a change in medication and a change in stimulation, the degree of treatment change may factor into the probability of a critical events origin.

Critical events that are life threatening and show a trend of negative change within the post treatment change window may be immediately flagged and may trigger a required action regardless of other outcomes 1181. If treatment change is only a stimulation change, then the stimulation may be reverted to a previous treatment if able 1182.

If a medication change is included, and if it is identified that a critical event is more likely the result of a known medication side effect, then the stimulation may be maintained and alerts may be sent to the patient, caregivers, physician and rep through connected apps and portals 1183. For each medication, expected effects and side effects that align with recorded critical events are flagged.

FIG. 12 illustrates, by way of example and not limitation, a method for identifying and addressing patient deterioration. At 1284 the patient's symptoms and side effects are tracked over time via a system of direct patient feedback and data collection through multiple wearables. Symptoms that are redundant may be averaged together in a weighted manner. The trend in symptom and side effects are reviewed through a series of rolling averages meant to identify trends of change that are occurring over different timespans 1285. By way of example and not limitation, rolling averages may be determined for timespans of 1 month, 3 months, 6 months, 1 year, 3 years, and 5 years. At 1286 it is determined whether there is a sudden decline. A sudden decline in a 1-3-month window most likely indicates a treatment change or a significant acute event. If there is a sudden decline, it is determined whether the data indicates a treatment change 1287. If there is no treatment change, then it may be determined whether there are other reasons for the change in the symptoms or side effects 1288. Examples of such reasons my include significant life events, medical reasons or emotional reasons. If a medical change has occurred outside of the condition being treated (e.g., DBS treating Parkinson's Disease), then it may be determined if cross interaction could be occurring and can be addressed such as medication interaction. A potential cause may be flagged and the physician may be alerted or otherwise notified. If, at 1287, it is determined that there has been a treatment change, then corrective action may be taken. If there is a longer treatment decline (e.g., a decline observable over 3-5-year windows, such declines more likely represent a significant deterioration in the patient's condition (e.g., Parkinson's Disease). If the patient has not been seen lately, then updated programming may be recommended to the physician and connected apps to attempt to recalibrate stimulation space. Earlier data points from a “healthier” state may be excluded from consideration of best new stimulation location.

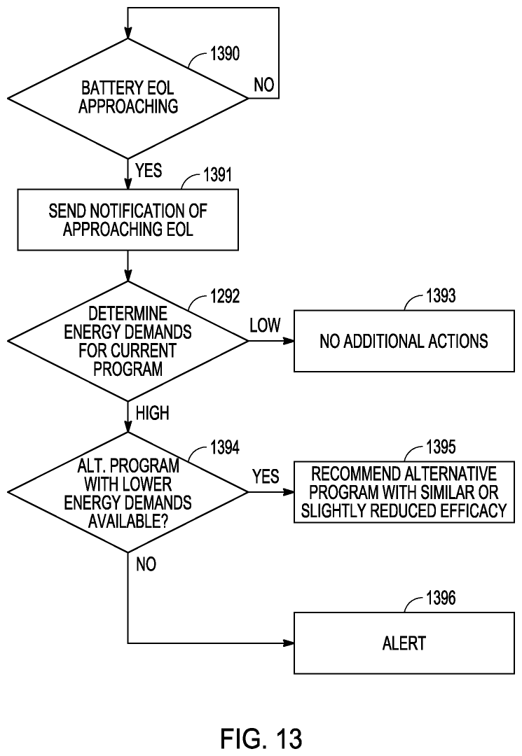

FIG. 13 illustrates, by way of example and not limitation, a method for identifying and addressing a need for a stimulator replacement. By way of example and not limitation, it may be determined whether a battery end of life (EOL) is approaching 1390. For example, primary cell battery notifications indicating that the battery is approaching end of life may be shown on the remote control to let the patient know that they need to schedule a replacement of the neurostimulator. Notifications of the approaching EOL may be sent at 1391. For example, this notification may be sent to all patient connected apps as well as the physician and rep portals to inform them of the event. At 1392, it is determined whether the energy demands for the current program are low or high. If the energy demands are low, then no additional actions may need to be taken by the system 1393 as the notifications have already been sent. If the energy demands for the current program are high, then it may be determined whether there is an alternative program with lower energy demands available 1394. If there is such a program, the system may recommend the alternative program with a similar or even slightly reduced efficacy 1395. If there is no such program that has lower energy demands, the system may send an alert to the physician, patient, device rep indicating that the battery is approaching EOL and that energy usage is high, such that a replacement should be performed.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
using a medical device configured to treat a condition by delivering a therapy to a patient, wherein the therapy is at least partially defined using a parameter set; and
using a processing system to:
determine that at least one event experienced by the patient is at least one critical event, each of the at least one critical event being one of a plurality of predefined critical events;
determine a severity of each of the at least one critical event, and weight each of the at least one critical event based on the determined severity to provide a corresponding at least one weighted critical event;
sum the at least one weighted critical event to provide a sum of at least one weighted critical event;
determine an overall clinical priority for the patient based at least in part on the sum, and weight the patient based on the determined overall clinical priority to provide a weighted patient priority;
select a communication technique from a plurality of available communication techniques for use to communicate the at least one critical event, the communication technique being selected based on the weighted patient priority; and
communicate the critical event using the selected communication technique.

2. The method of claim 1, wherein the processing system is further used to adjust the weighted patient priority based on whether corrective action is successfully performed by the medical device to address the at least one critical event, and the communication technique is selected based on the adjusted weighted patient priority.

3. The method of claim 2, further comprising performing the corrective action to address the at least one critical event, wherein the corrective action includes revising a current program, reverting to a prior program, replacing the current program with another program, or toggling programs.

4. The method of claim 1, wherein the medical device includes a deep brain stimulator (DBS) and the therapy includes a DBS therapy.

5. The method of claim 4, wherein the at least one critical event is specific to the condition of the patient, specific to the DBS therapy, or specific to the medical device used to deliver the therapy.

6. The method of claim 1, wherein the severity of each of the at least one critical event is determined using a plurality of weight factors that include at least two of the following:
whether the corresponding critical event is due to a newly recommended stimulation setting that the patient applied remotely;
whether the corresponding critical event is potentially life threatening;
whether the corresponding critical event is anticipated;
the regularity of an occurrence for the corresponding critical event based on a pre-assessed normal occurrence of the critical event for the patient; or
a comparison of a severity score for the corresponding critical event to a pre-assessed normal for the patient.

7. The method of claim 1, wherein the overall clinical priority for the patient is determined using a combination of the sum, as well as at least one of:
a determination that one or more of the at last one critical event is likely related to a change in medication or stimulation; and
when the patient is scheduled for a clinical visit.

8. The method of claim 1, wherein the weighted patient priority is determined to be high based on a single weighted critical event or based on a combination of two or more weighted critical events.

9. The method of claim 1, further comprising monitoring for the at least one event within an observation window to set for a time after a treatment change.

10. The method of claim 1, wherein the determined overall clinical priority is attributable to:
a treatment change and a determination whether the at least one critical event is life threatening or non-life-threatening;
patient deterioration rather than the treatment change or an acute event; or
a need for replacing the medical device.

11. The method of claim 1, wherein the plurality of available communication techniques for use to communicate the at least one critical event includes at least two techniques selected from a phone call, a text message, an email message, phone alerts including at least one of badges, banners or sound, a patient-connected app, a device rep portal and a clinician/physician portal.

12. A non-transitory machine-readable medium including instructions, which when executed by a machine, cause the machine to perform a method comprising:
determining that at least one event experienced by a patient is at least one critical event, each of the at least one critical event being one of a plurality of predefined critical events, wherein the patient is receiving a therapy delivered using a medical device to treat a condition;
determining a severity of each of the at least one critical event, and weight each of the at least one critical event based on the determined severity to provide a corresponding at least one weighted critical event;
summing the at least one weighted critical event to provide a sum of at least one weighted critical event;
determining an overall clinical priority for the patient based at least in part on the sum, and weighting the patient based on the determined overall clinical priority to provide a weighted patient priority;
selecting a communication technique from a plurality of available communication techniques for use to communicate the at least one critical event, the communication technique being selected based on the weighted patient priority; and
communicating the critical event using the selected communication technique.

13. The non-transitory machine-readable medium of claim 12, further comprising adjusting the weighted patient priority based on whether corrective action is successfully performed by the medical device to address the at least one critical event, wherein the communication technique is selected based on the adjusted weighted patient priority.

14. The non-transitory machine-readable medium of claim 12, wherein the medical device includes a deep brain stimulator (DBS) and the therapy includes a DBS therapy.

15. The non-transitory machine-readable medium of claim 14, wherein the at least one critical event is specific to the condition of the patient, specific to the DBS therapy, or specific to the medical device used to deliver the therapy.

16. The non-transitory machine-readable medium of claim 12, wherein the severity of each of the at least one critical event is determined using a plurality of weight factors that include at least two of the following:
whether the corresponding critical event is due to a newly recommended stimulation setting that the patient applied remotely;
whether the corresponding critical event is potentially life threatening;
whether the corresponding critical event is anticipated;
the regularity of an occurrence for the corresponding critical event based on a pre-assessed normal occurrence of the critical event for the patient; or
a comparison of a severity score for the corresponding critical event to a pre-assessed normal for the patient.

17. The non-transitory machine-readable medium of claim 12, wherein the overall clinical priority for the patient is determined using a combination of the sum, as well as at least one of:
a determination that one or more of the at last one critical event is likely related to a change in medication or stimulation; and
when the patient is scheduled for a clinical visit.

18. The non-transitory machine-readable medium of claim 12, wherein the weighted patient priority is determined to be high based on a single weighted critical event or based on a combination of two or more weighted critical events.

19. A system, comprising:
at least one medical device configured to treat a condition by delivering a therapy to a patient, wherein the therapy is at least partially defined using a parameter set, the medical device includes a deep brain stimulator (DBS) and the therapy includes a DBS therapy; and
a processing system configured for use to:
determine that at least one event experienced by the patient is at least one critical event, each of the at least one critical event being one of a plurality of predefined critical events, wherein the at least one critical event is specific to the condition of the patient, specific to the DBS therapy, or to the medical device used to deliver the therapy;
determine a severity of each of the at least one critical event, and weight each of the at least one critical event based on the determined severity to provide a corresponding at least one weighted critical event;
sum the at least one weighted critical event to provide a sum of at least one weighted critical event;
determine an overall clinical priority for the patient based at least in part on the sum, and weight the patient based on the determined overall clinical priority to provide a weighted patient priority;
adjust the weighted patient priority when corrective action is successfully performed to address the at least one critical event, wherein the corrective action is performed by revising a current program for the medical device, reverting to a prior program for the medical device, replacing the current program with another program for the medical device, or toggling programs for the medical device;
select a communication technique from a plurality of available communication techniques for use to communicate the at least one critical event, the communication technique being selected based on the weighted patient priority; and
communicate the critical event using the selected communication technique.

20. The system of claim 19, wherein the processing system is further used to adjust the weighted patient priority based on whether corrective action is successfully performed by the medical device to address the at least one critical event, and the communication technique is selected based on the adjusted weighted patient priority.
